(19) United States
(12) Patent Application Publication
(10) Pub. No.: US 2010/0010453 A1
(43) Pub. Date: Jan. 14, 2010

(54) Lockable shield for a syringe

(75) Inventor: John Riemelmoser, South Australia (AU)

Correspondence Address: KLAUBER & JACKSON, 411 HACKENSACK AVENUE, HACKENSACK, NJ, 07601, US

(73) Assignee: SAFETY MEDICAL PRODUCTS LIMITED, Wayville (AU)

(21) Appl. No.: 11/794,787

(22) PCT Filed: Jan. 6, 2006
(86) PCT No.: PCT/AU2006/000007

§ 371 Date: Aug. 12, 2009

(30) Foreign Application Priority Data
Jan. 7, 2005 (AU) 2005900045

(51) Int. Cl. A61M 5/32

(52) U.S. Cl. 604/198

(57) ABSTRACT

The invention is a shield (11) for a syringe (10) with a needle (18) where the shield (11) is moveable between a retracted position where the syringe (10) and needle (18) can be used and a shielding position where at least the tip of the needle (18) is covered to prevent accidental needle stick injuries, comprising a shield (11) slidably attached to the body of the syringe (10) that can be moved between retracted and shielding positions to at least shield the tip of the syringe needle (18) and locking means (12) comprising at least one forward facing resilient prong (15) located with respect to the syringe (10) and a projection (16) on the shield (11) whereby the projection (16) can deflect the prong (15) to pass over it, but abuts against the moveable end of the prong (15) to thereby prevent rearward movement of the shield (11).

### FIELD OF INVENTION

This invention relates to a syringe with a needle protection system and in particular to an arrangement that ensures that the tip of the needle at least can be covered by a sliding shield to thereby minimise the possibility of needle stick injuries.

### BACKGROUND OF THE INVENTION

The need to prevent injuries resulting from accidental contact with syringe needles is well-known. An increasing area of need is to prevent accidental needle stick injuries from the handling of syringes by medical personnel, either following the use of the syringe or after filling when it is not used immediately.

Accordingly, it is an object of this invention to provide a simple means whereby after use of a syringe, at least the tip of the needle can be covered by a shield to minimise the possibility of a needle stick injury.

### BRIEF DESCRIPTION OF THE INVENTION

In one form, the invention comprises a shield for a syringe with a needle that is moveable between a retracted position where the syringe and needle can be used and a shielding position where at least the tip of the needle is covered to prevent accidental needle stick injuries comprising;

a shield slidably attached to the body of the syringe that can be moved between said retracted and shielding positions to at least shield the tip of the syringe needle, and

locking means comprising at least one forward facing resilient prong located with respect to said syringe and a projection on said shield whereby the projection can deflect said prong to pass over it, but abuts against the moveable end of the prong to thereby prevent rearward movement of the shield.

Preferably, a pair of prongs may be used on either side of the syringe body and the prongs may either be moulded as part of the body of the syringe or alternatively may comprise a separate moulding that can be later attached or secured to the body of syringe.

Preferably, the shield comprises an elongate tubular member that is slidable along the body of the syringe. The length of the shield is sufficient to ensure that the end of the shield covers or locates past or adjacent to the tip of the needle of the syringe.

Preferably, the shield is moulded from a plastic material that is transparent to allow a sufficiently clear view of the body of the syringe during its operation. The shield may also have a lens portion moulded along its length or adjacent a portion of the graduations on the syringe body to provide magnification.

The projection may be a circumferential ridge on the inside surface of the tubular shield and there may be further abutment projections provided acting between the shield and syringe body to limit the extent of movement of the shield. The shield can be moved into a locked position where it is unable to move rearwardly or further forwardly.

In a further aspect of this invention, the shield for a syringe needle that is moveable between a retracted position and a shielding position may comprise a shield that can be slid forward to at least cover the tip of the needle of the syringe and a locking means that locks the shield in its lower position wherein said locking means is manually releasable so that the shield can again be retracted rearwardly.

In this aspect of the invention, the shield can be deployed part way through a procedure to minimise the likelihood of needle stick injury and then again retracted to allow use of the syringe.

Preferably, the locking means comprises a forward facing resilient prong where a portion of the prong is exposed when the shield is moved into its forward locked position. This enables the prong to be depressed to thereby release the lock to allow the shield to slide rearwardly. It will be apparent to the skilled addressee that other resilient locking means incorporating either hinged plastic components or metal spring arrangements may be equally suited to this aspect of the invention.

As with the previous aspect of the invention, the releasable locking arrangement may be moulded to the external surface of the syringe body or alternately may comprise a separate moulding which is later secured to the body of the syringe.

### DESCRIPTION OF A PREFERRED EMBODIMENT

In order to further understand the scope of the invention, a preferred embodiment will be described however it will be realised that the scope of the invention is not to be restricted to the features of the embodiment.

In the drawings:

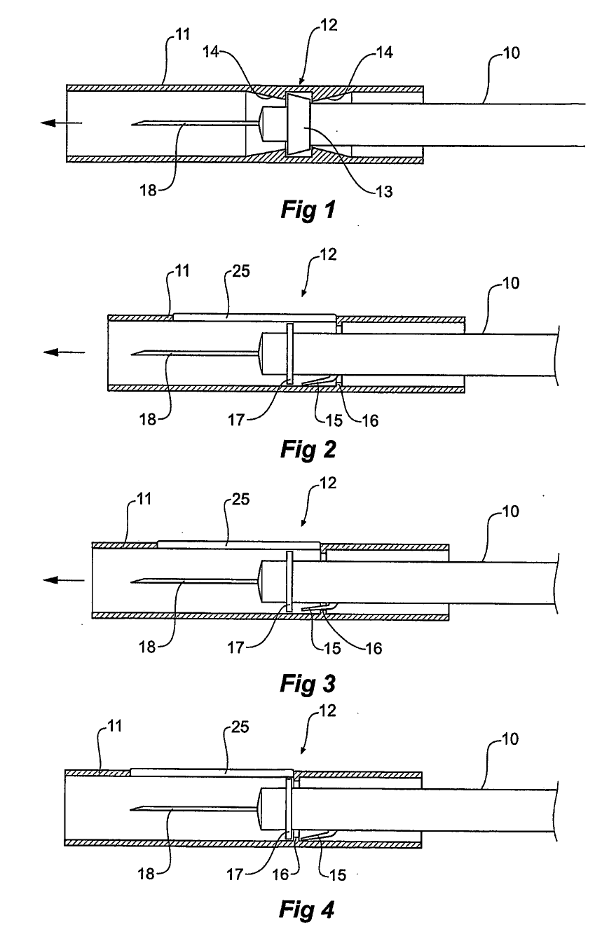

FIG. 1 shows an example of a prior art shield and locking arrangement,

FIGS. 2 to 4 show a first embodiment of the invention with the shield moving progressively to a locked position,

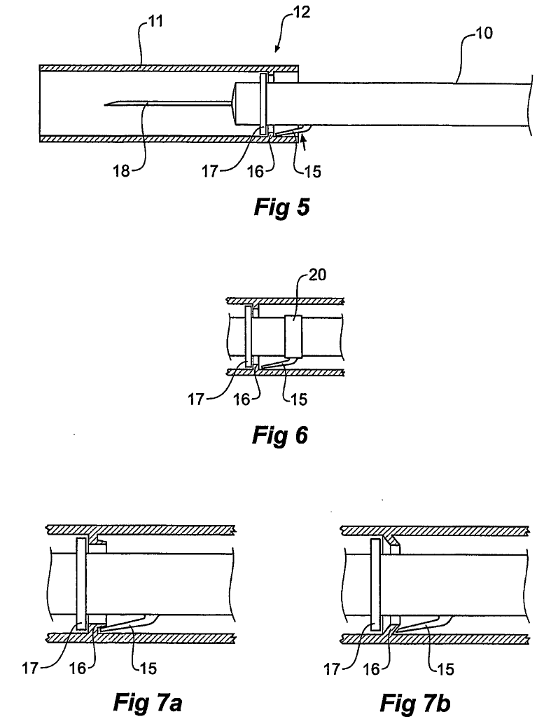

FIG. 5 shows a second embodiment of the invention,

FIG. 6 shows an alternative method of fixing a prong to a syringe, and

FIGS. 7a and 7b show alternative configurations for a projection on the shield.

Each of the drawings show a syringe body 10 in schematic form with a shield 11 moved to a forward position to cover a needle 18.

In the case of the prior art syringe shown in FIG. 1, the locking means 12 comprises a collar 13 on the end of the syringe body 10 which locates within a recess formed between a pair of circumferential ridges 14. The forward circumferential ridge 14 has a ramped surface which cooperates with the collar 13 to allow engagement of the locking means 12.

However, such an arrangement requires some considerable force to locate the shield in its locked position. Further, the locking means 12 can be overcome if sufficient rearward force is applied.

Also, the locking means 12 shown in FIG. 1 is not designed to be released and therefore once the shield 11 is locked in position, the syringe could not again be used which means that such an arrangement would not be suitable for temporary shielding of the needle.

FIGS. 2 to 4 show a first embodiment of the invention. The shield 11 is shown forward in its locked position in FIG. 4 and the locking means 12 comprises an elongate prong 15 and a projection 16. The prong 15 is moulded from a resilient material that is secured at one end. This results in the prong 15 being able to reflect resiliently towards the body 10.

The projection 16 is designed to ride over the top of the prong 15 and passes the prong 15 by resiliently deflecting it towards the body 10 (see FIG. 3). Once the projection 16 passes, the prong 15 springs outwardly to thereby block rearward movement of the shield 11 by abutment of the projection 16 against the end of the prong 15.

Further, any forced rearward movement of the shield 11 would result in the prong 15 buckling which in turn would jam the shield 11 with respect to the body 10. Only complete sheering of the prong 15 from its mounting would enable the shield 11 to be moved rearwardly. FIGS. 7a and 7b show alternative shapes for the projection 16 that have a recess (FIG. 7a) or are angled back (FIG. 7b) to more positively capture the prong 15.

A forward projection 17 limits forward movement of the shield 11. Also, in this embodiment, the shield 11 has sufficient rearward length from the projection 16 to ensure that the prong 15 remains covered when the shield 11 is in its locked position.

Another aspect of this invention is illustrated in FIG. 5 in which the locking means 12 is partially exposed with the shield 11 in its forward locked position. This enables manual release of the lock to allow the shield 11 to again move to its retracted position thereby exposing the needle 18.

In this embodiment, the locking means 12 again comprises a prong 15 and a projection 16. A projection 17 on the body 10 limits the forward movement of the shield 11.

However, in this embodiment, the shield 11 does not extend rearwardly from the projection 16 to fully cover the prong 15 in its locked position. Accordingly, the prong 15 can be manually deflected to allow the projection 16 to move rearwardly over it to thereby release the shield 11.

In both of the embodiments shown in FIGS. 2 to 4, and 5, the prong is illustrated as being integrally moulded with the body 10. However, a separate moulding securable to the syringe body 10 as shown in FIG. 6 could enable a locking means to be attached to a standard syringe body. In this embodiment, the attachment is a ring 20 that has an aperture within which the end of the syringe can be inserted with one or two prongs 15. A projection 17 could also be moulded on the external surface of an extended ring 20. The attachment may be formed in two halves for securing to either side of a syringe body. Alternatively, the attachment may comprise only a single prong 15 which is attached to one side of the body.

Preferably the shield 11 is moulded from a clear polymeric material which enables the contents of the syringe and graduations on its external surface to be clearly visible. Further, a lens 25 may be moulded into part of the shield 11 to provide magnification of the syringe body. This may be particularly helpful in relation to reading graduations on the syringe.

As will be seen from the above description, the invention provides novel means of shielding a needle after use of the syringe. Also, an aspect of the invention enables the shield to be temporarily positioned and then withdrawn for later use of the syringe.

1. A shield for a syringe with a needle that is moveable between a retracted position where the syringe and needle can be used and a shielding position where at least the tip of the needle is covered to prevent accidental needle stick injuries comprising;
a shield slidably attached to the body of the syringe that can be moved between said retracted and shielding positions to at least shield the tip of the syringe needle, and
locking means comprising at least one forward facing resilient prong located with respect to said syringe and a projection on said shield whereby the projection can deflect said prong to pass over it, but abuts against the moveable end of the prong to thereby prevent rearward movement of the shield.

2. A shield according to claim 1 wherein a pair of prongs are located on said syringe.

3. A shield according to claim 1 wherein said prongs are integrally moulded with the body of the syringe.

4. A shield according to claim 1 wherein said prongs are attached to the body of the syringe.

5. A shield according to claim 1 wherein the shield comprises an elongate tube.

6. A shield according to claim 5 wherein said elongate tube is transparent.

7. A shield according to claim 6 wherein a portion of said tube comprises an elongate lens portion.

8. A shield according to claim 5 wherein said projection comprises a circumferential ridge on the inside surface of the elongate tube.

9. A shield according to claim 8 wherein said projection has an angled or recessed surface, against which said prong abuts, that directs the tip of said prong towards the inside surface of the elongate tube.

10. A shield according to claim 1 comprising a further abutment projection on the syringe against which the projection on the shield abuts to limit movement of the shield when in its shielding position.

11. A shield according to claim 1 wherein the shield covers the prong when in the fully forward shielding position.

12. A shield according to claim 1 wherein a portion of said prong is exposed, sufficient to manually deflect it to allow said prong to pass said projection, when said shield is in its fully forward shielding position to allow manual retraction of said shield.

13. (canceled)

14. A shield according to claim 2 wherein said prongs are integrally moulded with the body of the syringe.

15. A shield according to claim 2 wherein said prongs are attached to the body of the syringe.

16. A shield according to claim 2 wherein the shield comprises an elongate tube.

17. A shield according to claim 2 comprising a further abutment projection on the syringe against which the projection on the shield abuts to limit movement of the shield when in its shielding position.

18. A shield according to claim 9 comprising a further abutment projection on the syringe against which the projection on the shield abuts to limit movement of the shield when in its shielding position.

19. A shield according to claim 2 wherein a portion of said prong is exposed, sufficient to manually deflect it to allow said prong to pass said projection, when said shield is in its fully forward shielding position to allow manual retraction of said shield.

20. A shield according to claim 9 wherein a portion of said prong is exposed, sufficient to manually deflect it to allow said prong to pass said projection, when said shield is in its fully forward shielding position to allow manual retraction of said shield.
